(19) United States
(12) Patent Application Publication
(10) Pub. No.: US 2010/0159533 A1
(43) Pub. Date: Jun. 24, 2010

(54) SIMPLIFIED SAMPLE PREPARATION FOR RNA ANALYSIS

(75) Inventors: Doron Lipson, Chestnut Hill, MA (US); Tal Raz, Brookline, MA (US); John F. Thompson, Warwick, RI (US)

Correspondence Address: LANDO & ANASTASI, LLP, ONE MAIN STREET, SUITE 1100, CAMBRIDGE, MA, 02142, US

(73) Assignee: HELICOS BIOSCIENCES CORPORATION, Cambridge, MA (US)

(21) Appl. No.: 12/625,115

(22) Filed: Nov. 24, 2009

Related U.S. Application Data
(60) Provisional application No. 61/117,291, filed on Nov. 24, 2008.

(51) Int. Cl. C12P 19/34; C07H 21/02; C07H 21/04

(52) U.S. Cl. 435/91.51; 536/24.33; 536/23.1

(57) ABSTRACT

Methods and kits for selective preparing cDNA relatively free of sequences found in rRNA and subcellular RNAs are disclosed. The methods and kits utilize approximately 200 hexamer sequences which target messenger RNA. The methods and kits are useful in preparing samples for sequencing analysis, especially when performing single molecule sequencing by synthesis.

### CROSS-REFERENCE TO RELATED APPLICATIONS

This application claims priority to U.S. Patent Application Ser. No. 61/117,291, filed on Nov. 24, 2008, under 35 U.S.C. §119, the contents of which are hereby incorporated by reference in their entirety.

### BACKGROUND

Analysis of messenger RNA (mRNA) is widely used to understand numerous aspects of gene regulation, including splicing variants, expression levels, gene rearrangements, variable translocation start sites (TSS) and polymorphisms (SNPs). High density microarrays are commonly used to analyze mRNA levels. Some of the commercially available arrays have certain limitations based on the limited diversity of capture probes. Quantification of mRNA on microarrays is routinely carried out by comparing relative ratios of a sample mRNA to a predetermined value, or ratiometric analysis. Often times, ratiometric analysis does not allow the sensitivity necessary to monitor small changes in mRNA levels, which can be potentially biologically relevant, e.g., less than 2-fold changes.

Methods capable of detecting and identifying all naturally-occurring mRNAs without bias are desirable. Recently developed methods of high throughput sequencing have the potential to perform such analysis of mRNA. In examples of these sequencing methods, the RNA (total) or the mRNA must first be converted to complementary DNA (cDNA) using standard methods in the art. These sequencing methods are capable of detecting any cDNA, which is produced from the RNA. Some of the most abundant RNA in cells is ribosomal RNA (rRNA), which is a component of the translational machinery within a cell in the conversion of mRNA to proteins. Detection of rRNA sequences often complicates the mRNA analysis. RNA from subcellular organelles, such as mitochondria and chloroplast (plants), are also sources of RNA which potentially interfere with mRNA-specific analyses.

Independent of the cellular origin, the preparation of mRNA from total RNA is a time-consuming and sometimes difficult task. Generally, total RNA is run through a column or exposed to beads with an oligoT capture sequence to separate out the polyA containing fraction of mRNA. PolyA mRNA is the mature transcript form of mRNA. However, other versions of pre-mRNA without the polyA are not analyzed when this purification method is utilized. Since this method can be tedious and difficult to effectively scale to large numbers of samples, a simpler method of selectively reverse transcribing all variants of mRNA species from total RNA is desirable.

### SUMMARY

The present invention provides, at least in part, methods and kits for preparing complementary DNA (cDNA) relative free of sequences found in ribosomal RNA (rRNA) and/or RNA originating from subcellular organelles. In one embodiment, the method utilizes approximately 200 hexamer sequences which selectively target messenger RNA (mRNA). Thus, the methods and kits provided herein are useful in preparing mRNA samples for RNA analysis, including sequencing analysis (e.g., single molecule sequencing analysis), polymerase chain reaction (RT-PCR) analysis, array expression analysis, and gene expression analysis.

Accordingly, in one aspect, the invention features a method for priming mRNA during reverse transcription of an RNA sample (e.g., total RNA or polyA RNA) to cDNA. The method includes contacting the RNA sample with one or more primer oligonucleotides comprising one or more oligonucleotide sequences which hybridize specifically to the mRNA present in the RNA sample under conditions that allow selective reverse transcription of the mRNA in the RNA sample to occur. For example, the oligonucleotide sequences can comprise a set of random oligonucleotide sequences (e.g., sequences about 6 to 12 bases in length and/or having a G+C a content of less than 70% of the entire sequence length). In certain embodiments, the transcribed mRNA is relatively free (e.g., contains less than 20%, 15%, 10%, 5%, or 1%) of non-mRNA sequences, e.g., rRNA (e.g., human 28S RNA, 18S RNA, 5.8S RNA, or 5S RNA) and/or RNA originating from subcellular organelles (e.g., mitochondrial RNA or chloroplast RNA).

In another aspect, the invention features a kit for priming mRNA during reverse transcription of an RNA sample (e.g., total RNA or polyA RNA) to cDNA. The kit includes (optionally) a reverse transcriptase, and one or more primer oligonucleotides comprising one or more oligonucleotide sequences (e.g., a plurality of oligonucleotide sequences) which hybridize specifically to the mRNA present in the RNA sample under conditions that allow selective reverse transcription of the mRNA to occur. The kit may, optionally, include instructions for use.

Certain embodiments of the methods or kits of the present invention are set forth below.

In one embodiment, the oligonucleotide sequences of the primer oligonucleotides comprise a pre-selected mixture of random sequence hexamers. For example, each of the random hexamer sequences can comprise those sequences containing no greater than 4 C+G per hexamer. In other embodiments, each of the random hexamers has at least two differences (e.g., has at least 2 base edit distance) compared to the most homologous sequence found in rRNA (e.g., human 28S RNA, 18S RNA, 5.8S RNA, or 5S RNA), mitochondrial RNA or chloroplast RNA. In other embodiments, the random hexamers comprise a portion or all of the hexamer sequences provided in Table 1, when the sample is of human origin. For example, the oligonucleotide sequences (e.g., the random hexamers) can comprise at least 1, 10, 25, 50, 75, 100, 125, 150, 175, 200 or more the hexamer sequences provided in Table 1.

In another embodiment, the oligonucleotide sequences of the primer oligonucleotides can additionally have a moiety which anchors them to a substrate (e.g., a substrate comprising an epoxide). For example, the moiety can be an additional base sequence complementary to surface attached primers; or one member of a binding pair (e.g., biotin and streptavidin) with the other member of the binding pair anchored on the substrate. In another example, the moiety can comprise a 5′-amine, 5′-azide, or 5′-alkynyl.

In one embodiment, reverse transcription of the mRNA in the sample occurs in the presence of a reverse transcriptase and a plurality of nucleotides under appropriate conditions (e.g., salt, buffer, temperature conditions) to allow the transcription to occur. For example, the reverse transcription can occur at a temperature between 37° C. to 55° C., e.g., 42° C. to 45° C. Reverse transcriptase enzymes and kits are commercially available. In other embodiments, reverse transcription can occur in solution or in a solid support.

In another aspect, the invention features a pre-selected mixture of random hexamer sequences. For example, each of the random hexamer sequences can comprise those sequences containing no greater than 4 C+G per hexamer. In other embodiments, each of the random hexamers has at least two differences (e.g., has at least 2 base edit distance) compared to the most homologous sequence found in rRNA (e.g., human 28S RNA, 18S RNA, 5.8S RNA, or 5S RNA), mitochondrial RNA or chloroplast RNA. In other embodiments, the random hexamers comprise a portion or all of the hexamer sequences provided in Table 1, when the sample is of human origin. For example, the random hexamers can comprise at least 1, 10, 25, 50, 75, 100, 125, 150, 175, 200 or more the hexamer sequences provided in Table 1. The random hexamer mixture can be stored in a suitable container or vial for distribution or shipping.

All publications, patent applications, patents, and other references mentioned herein are incorporated by reference in their entirety.

Other features, objects, and advantages of the invention will be apparent from the description and drawings, and from the claims.

### DETAILED DESCRIPTION

In order to obtain an accurate representation of RNAs present in a sample, priming of the reverse transcription reaction to make cDNA using random sequence hexamer oligonucleotides is commonly performed. Alternative methods using oligoT primers, hexamer and longer random sequence oligonucleotides, or both can also be practiced. When performed on total RNA, there is a significant contribution to the cDNA pool from ribosomal RNA (rRNA). In those cases, purification of mRNA-specific cDNA is generally required. To obviate this step, the present invention discloses a method of using a subset of random sequence hexamer oligonucleotides that selectively avoid priming of rRNA, mitochondrial RNA, and/or any other undesired subspecies.

The maximum numbers of random hexamer oligonucleotides includes 4096 different sequences, e.g., 4⁶. If the hexamer sequences which match perfectly with those sequences corresponding to human 28S RNA, 18S RNA, 5.8S RNA, and 5S RNA are removed from the set of 4096 possible sequences, there are 1248 non-rRNA sequences remaining. Additionally, in order to limit mis-priming by hexamers with one or two mismatches, it may be desirable to avoid hexamer sequences with high GC content. If only hexamers with a total of up to 4 total G+C bases are included, 1038 hexamer sequences remain. Further, our experiments with sequencing human mRNAs show that mitochondrial sequences also represent a significant component of the total RNA pool. It may be desirable to avoid hexamers with sequences in common with mitochondrial RNA. When transcribed mitochondrial sequences are omitted, 202 hexamers remain including 86 with 3 G+C and 55 with 4 G+C (see Table 1). When the set of 86 hexamer sequences with higher G+C is electronically tested against 38,991 human sequences in the Refseq database, 99.6% of transcripts would be reverse transcribed by at least one hexamer. The majority of the 156 transcripts that would not be transcribed are either hypothetical transcripts or less than 1 kb in length. If the entire set of 202 hexamers is used, only 17 hypothetical transcripts and no known transcripts of greater than 200 by would not be transcribed. The present example is for human genetics. However, similar analyses can be done on any sample from widely varying biological origins, including animal, plants, bacteria, fungi, protists, viruses, among others.

Selective amplification of mRNAs, including any other non-rRNA and non-mitochondrial RNAs, can be achieved using these pools of hexamer sequence oligonucleotides. Since priming is a necessary step for reverse transcription, this method would not require any additional steps and would eliminate one of the difficulties in generating cDNA. The resulting cDNA would then be used, e.g., for sequencing or measuring RNA levels (e.g., using a microarray).

Many different platforms exist for performing nucleic acid sequencing utilizing a polymerase and sequencing-by-synthesis. Sequencing reactions performed in solution may be separated using slab gel or capillary gel electrophoresis to determine the nucleic acid sequence. Other sequencing reactions may be performed directly on a solid support.

RNA may be fragmented before or after conversion to cDNA, RNA fragments are ideally <1 kB, and preferably 200-500b in length. Following fragmentation, the sample is anchored to a surface in preparation for sequencing. Additional modifications may or may not be necessary. However, one method involves attachment of a defined sequence onto each of the fragments generated. Defined sequences may be added onto either the 5′ or 3′ end, typically, at the 3′ end. Defined sequences added to the 5′ end are generally done by ligation-based methods. Such sequence can be used to attach a sequencing primer binding site and/or to enable anchoring of the fragments via hybridization. Alternatively, the fragments may be labeled in such a way as to provide anchoring to the surface via direct or indirect mechanisms, e.g., direct refers to covalent attachment and indirect refers to anchoring via a binding pair. The defined sequence may be generally a single, unique oligonucleotide sequence comprised of 2 or more bases attached to all fragments, or a homopolymeric sequence comprised of only a single base. Generally the sequence will be 20-70 bases in length, preferably 30-50 bases.

A ligase can be used to attach a unique oligonucleotide sequence to the RNA fragments. The ligation may be blunt-ended or via overhanging ends. Ligation may also be via single stranded to single stranded using, for example, CircLigase™, RNA ligase or DNA ligase.

A terminal deoxynucleotidyl transferase or polyA polymerase can be used to add homopolymeric sequences. A single nucleotide, dATP or ATP, can be used to produce the homopolymeric tail. Control of the average length of poly A's added is by a reaction control of the molar excess of (d)NTP over the fragment 3′-ends.

Additionally in one method, samples from many different sources are mixed and analyzed together. In this case, the sequences used to anchor the fragments to a surface may also be encoded in order to be able to discriminate the sample from which the sequences are obtained. The sequence may include a series of bases, e.g., 4-6 bases, which permit sample identification, e.g., a nucleic acid barcode.

Optionally, the sequences necessary for anchoring to the surface and/or priming, (e.g., a polyA when using oligoT coated substrates), may be appended directly onto the hexamer oligonucleotide. Generally, an additional base sequence would be added to the 5′-end of the hexamer. Functional moieties for indirect or direct anchoring may also be added to the 5′-end of the primers, e.g., biotin or amines, wherein the biotin would enable indirect attachment to a streptavidin-coated surface and the amine would enable direct (covalent) attachment to a substrate coated with epoxides. Other modes of direct attachment involve using an azide and an alkynyl moiety.

Once fragments are end-labeled and anchored to a surface, at least four major high-throughput sequencing platforms are currently available, including the Genome Sequencers from Roche/454 Life Sciences (Margulies et al. (2005) Nature, 437:376-380; U.S. Pat. Nos. 6,274,320; 6,258,568; 6,210,891), the 1G Analyzer from lIIlumina/Solexa (Bennett et al., (2005) Pharmacogenomics, 6:373-382), the SOliD system from Applied Biosystems (solid.appliedbiosystems.com), and the Heliscope™ system from Helicos Bio sciences (see, e.g., U.S. Patent App. Pub. No. 2007/0070349, the entire disclosure of which is hereby incorporated herein by reference for all purposes). Although these new technologies are significantly cheaper compared to the traditional methods, such as gel/capillary Gilbert-Sanger sequencing, the sequence reads produced by the new technologies are generally much shorter (−25-40 vs. −500-700 bases). For example, the average read lengths on the four major platforms are currently as follows: Roche/454, 250 bases (depending on the organism); Illumina/Solexa, 25 bases; SoliD, 35 bases; Heliscope, 25 bases.

An example of asynchronous single molecule sequencing-by-synthesis is described. Oligonucleotides 30-50 bases in length can be covalently anchored at the 5′ end to glass cover slips. These anchored strands perform two functions. First, they act as capture sites for the target template strands, if the templates are configured with capture tails complementary to the surface bound oligonucleotides. They can also act as primers for the template-directed primer extension that forms the basis of the sequence reading. The capture primers are at a fixed position site for sequence determination. Each cycle consists of adding the polymerase-labeled nucleotide analog mixture, rinsing, optically imaging the field containing millions of active primer template duplexes, and chemically cleaving the dye-linker to remove the dye. The labeled nucleotides are added either individually in a cycle or if the detectable moiety is spectrally resolvable more than one nucleotide can be added per cycle. The nucleotide analogs are such that they add only once per strand/cycle, e.g., a reversible terminator may be used or reaction conditions which on average add only a single dNTP analog. The cycle (synthesis, detection, and dye removal) is repeated up to 25, 50, 100 times and, possibly, more.

The real-time single molecule sequencing-by-synthesis technologies rely on the detection of fluorescent nucleotides as they are incorporated into a nascent strand of DNA that is complementary to the template being sequenced. This type of detection depends, at least in part, upon the ability of the imaging system to differentiate which of the four spectrally resolvable fluorescent nucleotides in the polymerase-labeled nucleotide mixture incorporates as the polymerase copies the template in near real time.

The illustrative claims appended hereto are intended to form part of the specification as though fully reproduced therein. Additionally, the sequences below may be chemically synthesized, e.g., artificially produced, or may be isolated or produced using other suitable methods.

TABLE 1
Hexamer Sequences for Human cDNA preparation

| | | | | |
|---|---|---|---|---|
| AAAACG | TGATTG | CTATCG | TGCTGA | GTCGAC |
| AAACGT | TGCTTT | CTCTTG | TGGACT | GTGCAC |
| AACGTA | TGTAAG | GACTTG | TGTCGT | GTGCGA |
| AAGATC | TGTCAT | GAGATC | TGTGAC | GTGGCA |
| AAGTGT | TGTTTC | GAGTCA | TGTGTC | GTGGTC |
| AAGTTC | TGTTTG | GAGTTG | TTAGCC | GTTCGG |
| ACGTAT | TTACGT | GATGGA | TTGAGG | GTTGCG |
| AGTTAG | TTATGC | GATTCG | TTGCGA | TAGCGC |
| ATACGA | TTCTGT | GCATAG | TTTGCG | TCACGG |
| ATACGT | TTGAGT | GCTGAT | AACCGG | TCGACG |
| ATACTG | TTGCTA | GGATGT | ACACGC | TCGCGT |
| ATAGAG | TTGTGT | GGCAAT | ACCGGT | TCGTCG |
| ATCGAT | TTTAGG | GGTATC | ACGACG | TGACCG |
| ATGTAC | TTTGCT | GGTCTA | ACGCGT | TGCCGT |
| ATTGCT | TTTTGC | GGTTAC | ACGCTG | TGCGCA |
| ATTGGT | AAGGTC | GTCAAG | ACGGAG | TGCGCT |
| CATGTA | ACGAAG | GTCATG | ACGTCC | TGGGCT |
| CGAAAT | ACGACT | GTCGTT | AGGCTG | TTGGGC |
| CGAATT | ACGCTT | GTCTAG | ATGCGC | |
| CGTAAT | ACGGTT | GTGAGT | CACGCT | |
| CTGATA | ACTCGT | GTGGTT | CAGTGG | |
| GACATA | ACTGGA | GTGTAC | CATGCG | |
| GAGATA | AGACTG | GTTAGC | CCGGTT | |
| GAGTTA | AGATCG | GTTCTG | CCTGTG | |
| GGATAT | AGGATG | GTTGTG | CGACAG | |
| GGTATT | AGGGTA | GTTTGC | CGCGTA | |
| GGTTAT | AGGTAC | TAAGCG | CGCGTT | |
| GTAATG | AGTGCA | TACGCT | CGGATG | |
| GTACAA | AGTGGT | TACGTC | CGGTCT | |
| GTAGAT | AGTTCG | TAGTGC | CGTGAC | |
| GTATCT | ATGCTG | TATCGC | CGTGCT | |
| GTATTC | ATGGAC | TATGCG | CGTGGT | |
| GTCTTA | CAGACT | TATGGG | CTAGCG | |
| GTTAGA | CAGGTA | TCAGCA | CTCTGC | |
| GTTTTG | CAGTGT | TCAGGT | CTTGAG | |
| TAACGT | CATACG | TCAGTG | GACACC | |
| TACGAT | CCAGAT | TCCAGA | GACTGC | |
| TACTGT | CCAGTT | TCGACT | GCAACG | |
| TAGATC | CGAGTT | TCGATC | GCGCTT | |
| TAGGTT | CGCTTA | TCGGTA | GCTCGA | |
| TAGTGT | CGGTAT | TCGTTC | GGCTAG | |
| TATACG | CGGTTA | TCGTTG | GGGACT | |
| TCTTGT | CGTGTA | TCTGTC | GGTACG | |
| TGAGAT | CGTTAG | TGAGGT | GGTAGG | |
| TGAGTA | CTAACG | TGCATC | GTAGGG | |
| TGATTC | CTACAG | TGCGTA | GTCAGG | |

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

All references cited herein are incorporated herein by reference in their entirety and for all purposes to the same extent as if each individual publication or patent or patent application was specifically and individually indicated to be incorporated by reference in its entirety for all purposes.

### EQUIVALENTS

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

We claim:

1. A method for selective priming during reverse transcription of RNA to cDNA, comprising contacting an RNA sample with one or more primer oligonucleotides, wherein said primer oligonucleotides are comprised of one or more oligonucleotide sequences which hybridize specifically to mRNA.

2. The method of claim 1, wherein the RNA sample is total RNA or polyA RNA.

3. The method of claim 1, wherein the oligonucleotide sequences comprise a set of random sequence oligonucleotides.

4. The method of claim 3, wherein the oligonucleotide sequences are 6-12 bases in length.

5. The method of claim 4, wherein the G+C content of the oligonucleotide sequences is less than 70% of the entire sequence length.

6. The method of claim 1, wherein the oligonucleotide sequences are a subset of random sequence hexamers.

7. The method of claim 6, wherein the oligonucleotide sequences comprises those sequences containing no greater than 4 C+G's.

8. The method of claim 6, wherein the hexamers are comprised of those sequences provided in Table 1 when the sample is of human origin.

9. The method of claim 6, wherein the oligonucleotide sequences are at least 2 base edit distance to nearest sequences found in rRNA, mitochondrial RNA or chloroplast RNA.

10. The method of claim 9, wherein the rRNA is human 28S RNA, 18S RNA, 5.8S RNA, or 5S RNA.

11. The method of claim 1, wherein the oligonucleotide sequences additionally have a moiety which anchors to the substrate.

12. The method of claim 11, wherein the moiety is additional base sequence complementary to surface attached primers.

13. The method of claim 11, wherein the moiety is one member of a binding pair, the other member of the binding pair be anchored on the substrate.

14. The method of claim 13, wherein the binding pair is biotin and streptavidin.

15. The method of claim 11, wherein the moiety comprises a 5′-amine, 5′-azide, or 5′-alkynyl.

16. The method of claim 11, wherein the substrate comprises an epoxide.

17. A kit for selective priming during reverse transcription of RNA to cDNA, comprising one or more primer oligonucleotides, wherein said primer oligonucleotides are comprised of one or more oligonucleotide sequences which hybridize specifically to mRNA.

18. A pre-selected mixture of random hexamer sequences, comprising a plurality of hexamer sequences comprising those sequences containing no greater than 4 C+G per hexamer; or a plurality of hexamer sequences provided in Table 1 when the sample is of human origin.

19. The mixture of claim 18, wherein the random hexamer sequences comprise a portion or all of the hexamer sequences provided in Table 1.
